(19) United States
(12) Patent Application Publication
(10) Pub. No.: US 2024/0217925 A1
(43) Pub. Date: Jul. 4, 2024

(54) Method for producing methionine

(71) Applicant: ADISSEO FRANCE S.A.S., ANTONY (FR)

(72) Inventors: Ana PINHEIRO, CHASSE-SUR-RHÔNE (FR); Didier MORVAN, MORNANT (FR); Nicolas CAPELLE, CORBAS (FR)

(21) Appl. No.: 18/289,196

(22) PCT Filed: May 20, 2022
(86) PCT No.: PCT/FR2022/050958

§ 371 Date: Nov. 1, 2023

(30) Foreign Application Priority Data
May 20, 2021 (FR) 21/05274

(51) Int. Cl. C07C 319/12; C07C 391/00

(52) U.S. Cl. CPC C07C 319/12; C07C 391/00

(57) ABSTRACT

The invention concerns a method for alkaline hydrolysis of 5-(2-methylmercaptoethyl)hydantoin of formula (1)

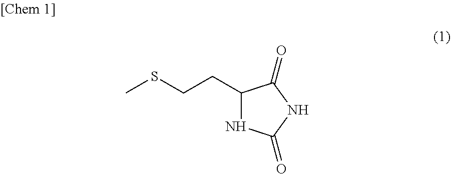

or of its seleniated equivalent, 5-(2-methylselenoethyl)hydantoin, into a methionine salt or a selenomethionine salt, respectively,
said method being carried out in a basic aqueous medium, in a reactive stripping packed column, as well as a method for producing the methionine and the selenomethionine implementing it.

The invention relates to an improvement of a method for producing methionine, in the form of salt or in the free form, or its seleniated analogue (selenomethionine), in the form of salt or in the free form, from the precursors 5-(2-methylmercaptoethyl)hydantoin for methionine and 5-(2-methylselenoethyl)hydantoin for selenomethionine.

The size of the methionine market no longer needs to be presented, particularly in animal nutrition, and its production methods are still the subject of numerous developments, with a view to even more cost-effective methods, but also less polluting and less energy-consuming. The seleniated derivatives of methionine are also constituents of major interest in animal nutrition.

The preparation of methionine may be practiced by different methods involving various synthesis intermediates, and in particular 5-(2-methylmercaptoethyl)hydantoin. This compound corresponds to the following formula (1):

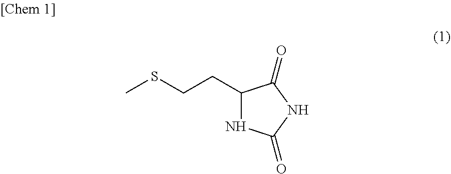

and its synthesis is widely documented (see for example U.S. Pat. No. 5,990,349A).

The 5-(2-methylmercaptoethyl) hydantoin may be hydrolyzed in an alkaline medium to obtain a methionine salt which is then neutralized into methionine, if necessary.

Thus, according to WO2013/030068A2, a method for continuously producing a methionine salt is known, this method comprising the alkaline hydrolysis of 5-(2-methylmercaptoethyl)hydantoin into methionine salt, conducted in a reaction-rectification plate column, preferably perforated plate column. The latter must satisfy characteristics of number of plates, overflow height, space between plates, diameter of the column compared to the overflow length and of ratio of the cross-section of the column to the gas flow surface, which have been determined very precisely, having the advantage of minimizing the formation of by-products such as methionine dipeptides.

An aqueous solution of hydantoin is introduced at the level of the upper plate of the column and an alkaline solution based on alkali metal carbonate is fed at the level of a plate under the upper plate of the column. The heat required for the reaction is provided by a water vapor stream supplied to the bottom of the column.

The alkaline hydrolysis of methionine hydantoin leads to the formation of ammonia gas and carbon dioxide. It is a very fast process and one of the problems encountered by reactive rectification technology lies in untimely gas production which must be controlled by any means. On the one hand, it is necessary to control the speed of the hydrolysis reaction and on the other hand, it is necessary to provide for an evacuation of the formed gases and in particular of carbon dioxide, as they are released; indeed, the carbon dioxide in the reaction medium will produce carbonic acid causing a decrease in the pH of the medium and a drop in the selectivity for methionine.

The present invention provides a method for alkaline hydrolysis of methionine hydantoin using a reactive stripping technology to overcome these obstacles. For this purpose, the reaction is carried out, completely or partially, in a reactive stripping packed column.

The invention concerns a method for alkaline hydrolysis of 5-(2-methylmercaptoethyl)hydantoin of formula (1)

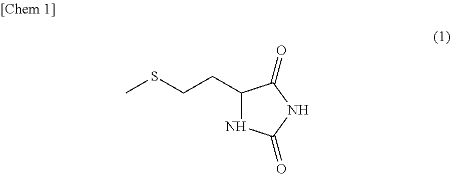

or of its seleniated equivalent, 5-(2-methylselenoethyl)hydantoin, into a methionine salt or a selenomethionine salt, respectively,
said method being conducted in a basic aqueous medium, in a reactive stripping packed column.

According to a variant of the invention, the aforementioned alkaline hydrolysis method is conducted in a basic aqueous medium, first in a reactive stripping packed column, then in at least one reactor, identical to or different from said reactive stripping packed column.

Before exposing the invention in more detail, the definition of some terms used in this text is given hereinafter.

The 5-(2-methylmercaptoethyl)hydantoin, or methionine hydantoin, corresponding to the aforementioned formula (1) may be in the form of a salt. It may be in a pure or a mixed state. A mixture may comprise one or several precursor(s) for the production of methionine, which will generally be the case when the invention falls in the scope of a method for the production of methionine on an industrial scale; methionine or a methionine salt, in particular when the hydrolysis of methionine hydantoin has already started; secondary products; as well as impurities. Of course, this definition applies identically to 5-(2-methylselenoethyl)hydantoin, as well as the entire description of this text.

By reactive stripping, or reactive distillation, or reactive rectification, it should be understood within the scope of the invention, the progress of the reaction, namely the alkaline hydrolysis of hydantoin into methionine salt (or selenomethionine salt) in the liquid phase and the separation from this phase of at least part of the produced gases, within the same column, called a reactive stripping, or reactive distillation, or reactive rectification column, which is a well-documented technology belonging to the general knowledge of a person skilled in the art.

The expressions “first in a reactive stripping packed column” and “then in at least one reactor” mentioned in a variant of a method of the invention, refer to a notion of order in the method, the hydantoin being according to the invention first treated in the reactive stripping packed column, then the resulting stream being afterwards treated in one reactor or several successive reactors. They do not, however, impart any limitation in the arrangement of the packed column and the reactor or reactors. Thus said packed column and the reactor may be in series, optionally separated by an enclosure for example. According to this variant of the invention, by reactive stripping, or reaction distillation, or reactive rectification column, it should be understood a column which may include different fillings, such as packings, plates, and any other filling, each capable of constituting a reactor. Thus it will be the case, when one or several reactor(s) is/are stripping columns disposed in the continuation the packed column, which will come down to a reactive stripping column, equipped with a packing in its upper portion and with at least one other filling in its lower portion.

As said before, the alkaline hydrolysis of methionine hydantoin is a reaction that is well known to those skilled in the art and, by “basic or alkaline medium”, is should be understood a medium whose pH makes it possible to hydrolyze the hydantoin into methionine salt; it is preferably of at least 8, preferably of at least 9; it may reach 13; even if it may vary with the evolution of the reaction, it is ideally maintained at this value of at least 8.

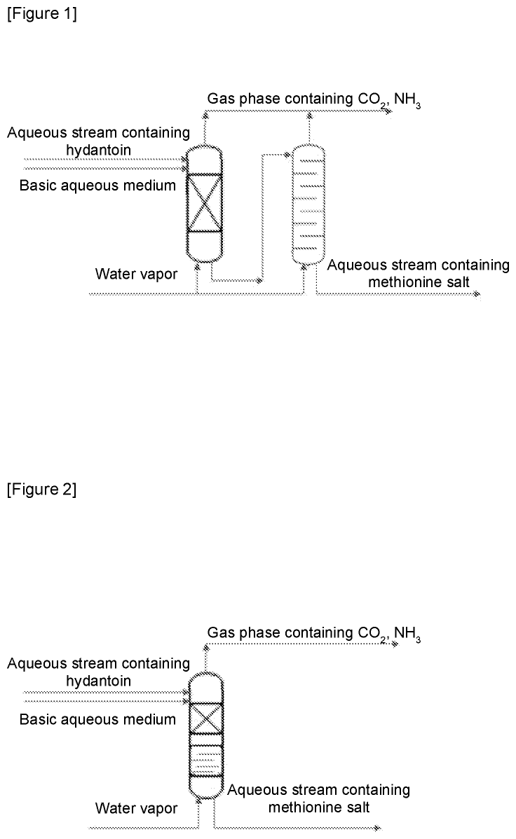

The present invention is described hereinafter in more detail, as well as preferred variants for implementing it and particular embodiments illustrated in the following FIGS. 1, 2, 3 and 5, FIG. 4 illustrating a method known from the prior art and provided for comparison:

FIG. 1 represents an implementation of the method of the invention in a reactive stripping packed column then in a reactor consisting of a reactive stripping plate column.

FIG. 2 represents an implementation of the method of the invention in a single and same column, including first a first section of reactive stripping packed column then a second section of reactive stripping plate column.

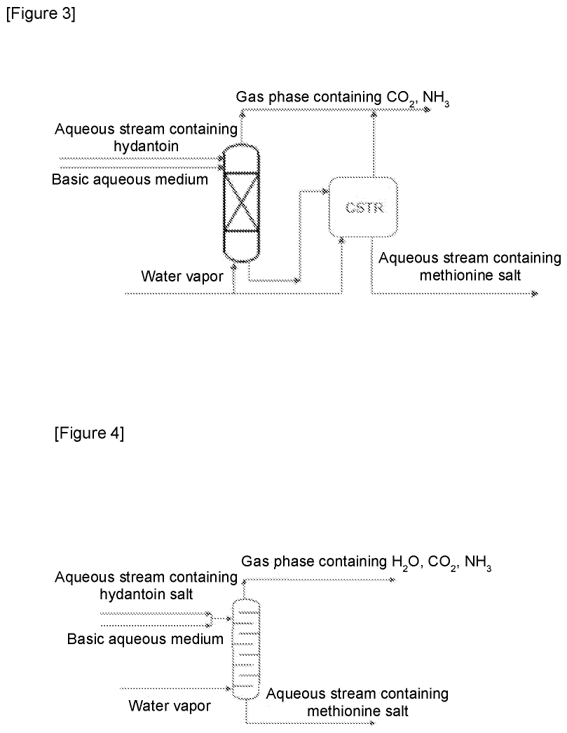

FIG. 3 represents an implementation of the method of the invention first in a reactive stripping packed column then in a CSTR reactor.

FIG. 4 represents, for comparison, an implementation of a hydrolysis method in a reactive stripping plate column according to one embodiment that does not form part of the invention.

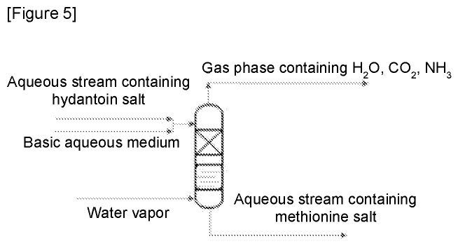

FIG. 5 represents an implementation of the method of the invention in a reactive stripping plate column then in a reactive stripping packed column.

According to the method of the invention, the alkaline hydrolysis of hydantoin is performed in a reactive stripping packed column.

Any type of reactive stripping packed column may be used. Thus, the packing may be ordered, also called structured, or random packing.

The packing consists of elements made of one or several material(s) capable of withstanding the reaction conditions. Advantageously, they are selected from metals and stainless and corrosion-resistant metal alloys such as zirconium, tantalum, titanium, stainless steels, Hastelloy® alloys; plastics such as; fluoropolymers like polytetrafluoroethylenes (PTFE), perfluoroalkoxy (PFA); the ceramics.

The packing of the column may consist of a succession of at least two different packings. They may be different, structured and/or random in their arrangement, and/or in the nature of their elements.

The reactive stripping packed column is conventionally equipped with an inlet at the bottom of the column of a hot gas of the water vapor type which moves in the upward direction. Alternatively, it may be provided, in its lower portion, with a heating means and equipped with at least one inlet of an inert gas, such as nitrogen, which is brought to the required temperature by said heating means and rises in the column. A person skilled in the art is able to adapt the circulation conditions of a hot gas stream with a view to optimizing the alkaline hydrolysis.

Whether the packing is structured or random, the column may be equipped with any means favoring an optimal distribution of the streams over all the packing elements in order to optimize the residence time of the basic aqueous medium in the column containing the hydantoin while allowing rapid evacuation of the formed gases. These means may consist of distributors and collectors, which are means conventionally employed in distillation technology. It may also be provided with any other known means facilitating the implementation of the reaction.

When the alkaline hydrolysis of hydantoin is not completely or almost completely concluded in a so-called reactive stripping packed column, that is to say when it is only partially carried out in said column, the alkaline hydrolysis may then be continued in one or several reactor(s).

The reactor(s) may be selected from reactive stripping columns, for example from any type of reactive stripping packed, plate, or bubble column.

The reactor(s) may also be selected from Continuous Stirred Tank Reactors (CSTR) and Plug Flow Reactors (PFR). They are preferably selected from reactors suitable for continuous methods, such as plug-flow reactors.

When several reactors are used, they may be identical or different. By different reactors, it should be understood for example reactors selected from reactive stripping columns, whose fillings are different in terms of nature (packed column, plate column, bubble column, etc.), in terms of materials, etc. Also, it should be understood that at least one reactor is selected from reactive stripping columns and the other is selected from CSTR or PFR reactors, or that the reactors are selected from CSTR or PFR reactors.

According to the invention, the method may be completed by any finishing step of the reaction in one or several additional column(s).

The basic aqueous medium comprises an alkaline hydrolysis reagent which is preferably an alkali metal salt. It is advantageously selected from alkali metal carbonates, alkali metal hydroxides and any mixture of said carbonates and hydroxides, and even better from potassium carbonate, potassium hydrogen carbonate, sodium hydroxide, potassium hydroxide and mixtures thereof.

The method of the invention is applied to 5-(2-methylmercaptoethyl)hydantoin which is supplied in the form of a stream containing only hydantoin, but which may also comprise one or several compound(s) selected from precursors of 5-(2-methylmercaptoethyl)hydantoin, methionine, salts and peptides, especially methionine dipeptides and derivatives, such as methionine hydantoate salts, 2-amino-4-methylthio-butyramide, 4-methylthio-2-ureido-butyramide, formate salts, 2-hydroxy-4-methylthio-butyrate salts, and impurities.

According to one variant, the method is carried out at a temperature from 160 to 200° C., preferably from 170 to 190° C. When the method is carried out first in a reactive stripping packed column, then in one or several reactor(s), the temperatures in said column and in the reactor or reactors may be identical or different. At a temperature lower than 160° C., the reaction does not occur or is insufficient. Above 200° C., the methionine salt selectivity decreases, in particular methionine dipeptides are formed in unacceptable quantities.

According to one variant, the method is carried out at a pressure from 7 to 15 bars, preferably from 8 to 12 bars. When the method is carried out first in a reactive stripping packed column, then in one or several reactor(s), the pressures in said column and in the reactor or reactors may be identical or different. A main advantage of the invention is to overcome the drawbacks related to the speed of the hydrolysis reaction and to optimize its yield and its selectivity for methionine salt (or selenomethionine salt). To this end, the residence time of the liquid reaction stream in the reactive stripping packed column is preferably of at least 1 minute, preferably of the order of 1 to 3 minutes. When the method is carried out in two stages, namely in a reactive stripping packed column and in one or several reactor(s), the residence time of the liquid reaction stream in the whole assembly of the reactive stripping packed column and the reactor or reactors, may vary from 6 to 10 minutes.

The method of the invention is adapted for a continuous mode or not. Advantageously, it is implemented continuously.

FIGS. 1 to 3 and 5 exemplify variants of the invention.

According to FIG. 1, an aqueous solution of hydantoin, together with an aqueous solution of potassium carbonate are fed to the top of a reactive stripping packed column, and a water vapor stream is supplied through an inlet at the bottom of the column and circulates against the flow of the hydantoin solution. Carbon dioxide and ammonia gases are discharged from the top of said column. The reaction stream resulting from passing through the packed column is extracted at the bottom of the column to feed a reactive stripping plate column from the top and in which a water vapor stream circulates against the flow. An aqueous stream of methionine salt is recovered at the bottom of this column.

According to FIG. 2, the method is carried out in a reactive stripping column equipped with a packing in its upper portion and with plates in its lower portion. An aqueous solution of hydantoin and an aqueous solution of potassium carbonate are fed to the top of the reactive stripping column, and a water vapor stream is supplied from an inlet at the bottom of the reactive stripping column and circulates against the flow of the hydantoin solution over the entire length of the column. Carbon dioxide and ammonia gases are discharged from the top of the column and an aqueous stream of methionine salt is recovered from the bottom of the column.

According to FIG. 3, an aqueous solution of hydantoin, as well as an aqueous solution of potassium carbonate are fed to the top of a reactive stripping packed column, and a water vapor stream is supplied by an inlet at the bottom of the column and circulates against the flow of the hydantoin solution. Carbon dioxide and ammonia gases are discharged from the top of said column. The reaction stream resulting from passing through the packed column is extracted at the bottom of the column to feed a CSTR-type reactor which also includes a water vapor inlet. An aqueous stream of methionine salt is recovered from this reactor.

The invention also concerns a method for the production of methionine or selenomethionine, from 5-(2-methylmercaptoethyl)hydantoin, or its seleniated equivalent, 5-(2-methylselenoethyl)hydantoin, respectively, comprising the hydrolysis method as previously described.

To obtain methionine or selenomethionine in free form, this production method will comprise the neutralization of the methionine salt into methionine and of the selenomethionine salt into selenomethionine. This neutralization is well known, it is advantageously carried out in the presence of CO₂.

The alkaline hydrolysis method alone or integrated into a methionine or selenomethionine production method may be supplemented by any recycling and/or gas or heat recovery system for reuse in the method or for another enhancement.

The experimental part set out hereinbelow compares a method for alkaline hydrolysis of hydantoin of formula (1) conducted in a basic medium in a reactive stripping plate column as known from the state of the art according to Example 1, and in a reactive stripping column equipped with a structured packing on the upper section of the column and equipped with plates on the lower section in accordance with the invention according to Example 2, under the same conditions.

### Example 1: Reactive Stripping Plate Column

This example has been carried out on a reactive stripping pilot column, with a total height of 6 m, and a diameter of DN 50.

This column was equipped with plates according to the diagram represented in FIG. 4.

The column was fed continuously, at the overhead of the column, with 12 kg/h of an aqueous solution at 180° C., this flow rate corresponding to a residence time of the liquid phase of 10 min.

The aqueous feed solution consisted of:
19.3% (w/w) of 5-(2-methylmercaptoethyl)hydantoin+precursors
15.2% (w/w) of potassium carbonate

The stripping was performed by injecting 2 kg/h of water vapor at 190° C.

The column was operated at a pressure of 10.5 bars, with a temperature profile ranging from 180° C. at the overhead to 190° C. at the bottom of the column.

The composition of the liquid reaction mixture obtained at the bottom of the column under steady state conditions was as follows:
0.2% (w/w) of 5-(2-methylmercaptoethyl)hydantoin+ precursors
20.4% (w/w) of methionine salt (potassium methionine)
0.64% (w/w) of methionine dipeptides

These results correspond to a conversion of 99% of 5-(2-methylmercaptoethyl)-hydantoin [+ precursors], with a selectivity of 96% for methionine salt and selectivity of 4% for methionine dipeptides.

### Example 2—Reactive Stripping Plate Column then Packed Column

This example has been carried out on a reactive stripping pilot column, with a total height of 6 m, and a diameter of DN 50.

The column was equipped with structured packing on the top section of the column and with plates on the bottom section, according to the diagram represented in FIG. 5:

The column was fed continuously, at the overhead of the column, with 12 kg/h of an aqueous solution at 180° C., this flow rate corresponding to a total residence time of the liquid phase of 10 min, including 2 min of residence time in the packing section and 8 min of residence time in the plate section.

The aqueous feed solution consisted of:
19.3% (w/w) of 5-(2-methylmercaptoethyl)hydantoin+ precursors
15.2% (w/w) of potassium carbonate

The stripping was carried out by injecting 2 kg/h of steam at 190° C.

The column was operated at a pressure of 10.5 bars, with a temperature profile ranging from 180° C. at the overhead to 190° C. at the bottom of the column.

The composition of the liquid reaction mixture obtained at the bottom of the column under steady state conditions was as follows:
0.1% (w/w) of 5-(2-methylmercaptoethyl)hydantoin+ precursors
20.6% (w/w) of methionine salt (potassium methionine)
0.56% (w/w) of methionine dipeptides

These results correspond to a conversion of 99.5% of 5-(2-methylmercaptoethyl)-hydantoin [+ precursors], with a selectivity of 96.5% for methionine salt and selectivity of 3.5% for methionine dipeptides.

Compared to Example 1, in Example 2 according to the invention, an increase in the conversion of hydantoin, an increase in the selectivity for methionine salt and a decrease in the selectivity for methionine dipeptides are observed. On an industrial scale, these variations are of considerable interest.

1. A method for alkaline hydrolysis of 5-(2-methylmercaptoethyl)hydantoin of formula (1) 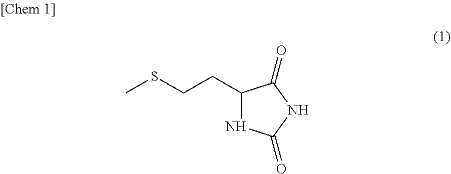
or of its seleniated equivalent, 5-(2-methylselenoethyl)hydantoin, into a methionine salt or a selenomethionine salt, respectively,
wherein it is carried out in a basic aqueous medium, in a reactive stripping packed column.

2. The method according to claim 1, wherein the reactive stripping packed column comprises one or more packings selected from structured and random packings.

3. The method according to claim 1, wherein the packing(s) of the reactive stripping packed column are made of one or several material(s) selected from metals and stainless and corrosion-resistant metal alloys such as zirconium, tantalum, titanium, stainless steels, Hastelloy® alloys; plastics such as; fluoropolymers like polytetrafluoroethylenes (PTFE), perfluoroalkoxy (PFA); the ceramics.

4. The method according to claim 1, wherein it is carried out in a basic aqueous medium, first in a reactive stripping packed column, then in at least one reactor.

5. The method according to claim 4, wherein said reactor or said reactors is/are selected from reactive stripping columns.

6. The method according to claim 5, wherein the reactor(s) is/are selected from reactive stripping packed columns, reactive stripping plate columns and reactive stripping bubble columns.

7. The method according to claim 4, wherein the reactor(s) is/are selected from Continuous Stirred Tank Reactors (CSTR) and Plug Flow Reactors (PFR).

8. The method according to claim 4, wherein it is carried out in a basic aqueous medium in a reactive stripping packed column, then in at least two successive reactors, or even more.

9. The method according to claim 1, wherein the basic aqueous medium comprises an alkaline hydrolysis reagent selected from alkali metal carbonates, alkali metal hydroxides and any mixture of said carbonates and hydroxides.

10. The method according to claim 1, wherein the alkaline hydrolysis reagent is selected from potassium carbonate, potassium hydrogen carbonate, sodium hydroxide, potassium hydroxide and mixtures thereof.

11. The method according to claim 1, wherein 5-(2-methylmercaptoethyl)hydantoin is supplied in the form of a stream further comprising one or several compound(s) selected from the precursors of 5-(2-methylmercaptoethyl)hydantoin, methionine, salts and peptides, especially dipeptides, of methionine and derivatives, such as hydantoate salts of methionine, 2-amino-4-(methylthio)-butyramide, 4-(methylthio)-2-ureido-butyramide, formate salts, 2-hydroxy-4-(methylthio)-butyrate salts, and impurities.

12. The method according to claim 1, wherein it is carried out at a temperature from 160 to 200° C., preferably from 170 to 190° C.

13. The method according to claim 1, wherein it is carried out at a pressure from 7 to 15 bars, preferably from 8 to 12 bars.

14. The method according to claim 1, wherein the residence time of the liquid reaction stream in the reactive stripping packed column is at least 1 minute, preferably from 1 to 3 minutes.

15. The method according to claim 5, wherein the residence time of the liquid reaction stream in the whole assembly of the reactive stripping packed column and the reactor(s) varies from 6 to 10 minutes.

16. The method for producing methionine from 5-(2-methylmercaptoethyl)hydantoin of formula (1) 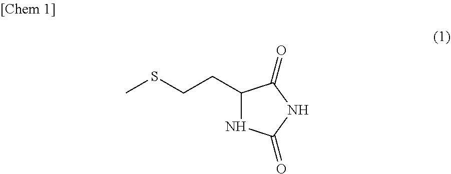
or selenomethionine from 5-(2-methylselenoethyl)hydantoin, comprising the method for hydrolysis of 5-(2-methylmercaptoethyl)hydantoin or 5-(2-methylselenoethyl)hydantoin as defined in claim 1.

17. The method according to claim 15, wherein it comprises neutralizing the methionine salt into methionine or the selenomethionine salt into selenomethionine.

18. The method according to claim 16, wherein the neutralization of the methionine salt or of the selenomethionine salt is performed in the presence of CO₂.
